(19) United States
(12) Patent Application Publication
(10) Pub. No.: US 2007/0172471 A1
(43) Pub. Date: Jul. 26, 2007

(54) Method for identifying an anti- streptococcal agent and its use for treating streptococcal infections

(75) Inventors: Lars Bjorck, Lund (SE); Heiko Herwald, Veberod (SE); Matthias Morgelin, Lund (SE); Wayne Russell, Kavlinge (SE); Anna Norrby-Teglund, Ronninge (SE); Lennart Lindbom, Stockholm (SE); Ulla Sollenberg, Stockholm (SE); Henning Cramer, Rodgale (DE); Hans Flodgaard, Hellerup (DK)

Correspondence Address: FOLEY AND LARDNER LLP;SUITE 500, 3000 K STREET NW, WASHINGTON, DC, 20007, US

(73) Assignee: Hansa Medical AB

(21) Appl. No.: 10/553,904

(22) PCT Filed: Apr. 23, 2004
(86) PCT No.: PCT/EP04/04429

§ 371 Date: Mar. 29, 2006

(30) Foreign Application Priority Data
Apr. 23, 2003 (GB) 0309246.7
Dec. 16, 2003 (GB) 0329112.7

(51) Int. Cl. C12Q 1/37; C12Q 1/18; A61K 38/48

(52) U.S. Cl. 424/94.63; 435/32; 435/23

(57) ABSTRACT

A method for identifying an anti-streptococcal agent, comprises: (a) providing, as a first component, an isolated streptococcal M protein or a functional variant thereof; (b) providing, as a second component, isolated fibrinogen or a functional variant thereof; (b) providing, as a third component, an isolated β₂ integrin or a functional variant thereof; (d) contacting said components with a test substance under conditions that would permit the components to interact in the absence of the test substance; and (e) determining whether the test substance inhibits the interaction between the components; thereby to determine whether a test substance is an anti-streptococcal agent.

### FIELD OF THE INVENTION

The invention relates to methods for identifying anti-streptococcal agents. The invention also relates to the use of such agents in the treatment of streptococcal infections.

### BACKGROUND TO THE INVENTION

Streptococcus pyogenes is one of the most common and important human bacterial pathogens. It causes relatively mild infections such as pharyngitis (strep throat) and impetigo, but also serious clinical conditions like rheumatic fever, post-streptococcal glomerulonephritis, necrotizing fasciitis, septicemia, and streptococcal toxic shock syndrome (STSS). Increases in the number of life-threatening systemic S. pyogenes infections have been reported worldwide since the late 1980s, and have attracted considerable attention and concern.

S. pyogenes expresses substantial amounts of M protein, α-helical coiled-coil surface proteins. M protein is a clinical virulence determinant of S. pyogenes which promotes the survival of the bacterium in human blood. Apart from being associated with the bacterial cell wall, M protein is also released from the surface by the action of a cysteine proteinase secreted by the bacteria.

Polymorphonuclear neutrophils (PMNs) are part of the first line of defence against bacterial infections. The recruitment of these cells from the bloodstream to an inflamed site involves their recognition of inflammatory mediators, their interaction with adhesion molecules of the vascular endothelium, and, finally, their migration across the endothelial barrier to the site of infection where PMNs phagocytize invading bacteria. Under physiological conditions non-activated PMNs circulate in the bloodstream. However, once activated by a chemotactic signal, they become adherent and begin to roll on the endothelium towards the site of infection, where they attach firmly to the endothelium and start to extravasate into the infected tissue. These adhesion processes involve the sequential up- and down-regulation of a number of different adhesion molecules both on PMNs and the endothelium, including integrins. Activated PMNs also release heparin-binding protein (HBP) from its intracellular storage. HBP is an inflammatory mediator that induces vascular leakage.

### SUMMARY OF THE INVENTION

The present inventors have shown that interactions between streptococcal M protein-fibrinogen complexes and β₂ integrins of PMNs cause activation of PMNs and release of heparin binding protein (HBP), thereby causing an inflammatory response. This interaction presents a novel target for the identification of anti-streptococcal agents, which can be used to block the interaction between streptococcal M protein-fibrinogen complexes and β₂ integrins thus preventing the activation of PMNs and therefore blocking the inflammatory response that would otherwise result.

In accordance with the present invention, there is thus provided a method for identifying an anti-streptococcal agent, which method comprises:

(a) providing, as a first component, an isolated streptococcal M protein or a functional variant thereof;

(b) providing, as a second component, isolated fibrinogen or a functional variant thereof;

(c) providing, as a third component, an isolated β₂ integrin or a functional variant thereof;

(d) contacting said components with a test substance under conditions that would permit the components to interact in the absence of the test substance; and

(e) determining whether the test substance inhibits the interaction between the components;

thereby to determine whether a test substance is an anti-streptococcal agent.

The invention also provides:

a method for identifying an anti-streptococcal agent, which method comprises:

(a) providing, as a first component, a streptococcal M protein or a functional variant thereof;

(b) providing, as a second component, fibrinogen or a functional variant thereof;

(c) providing, as a third component, one or more polymorphonuclear neutrophils (PMNs);

(d) contacting said components with a test substance under conditions that would permit the components to interact in the absence of the test substance; and

(e) monitoring any inhibition of the activation of PMNs; thereby to determine whether a test substance is an anti-streptococcal agent;

a test kit suitable for use in identifying a test substance which is capable of inhibiting the interaction between a streptococcal M protein or a functional variant thereof, fibrinogen and a functional variant thereof and a β₂ integrin or a functional variant thereof, which kit comprises:

(a) an isolated streptococcal M protein or a functional variant thereof;

(b) isolated fibrinogen or a functional variant thereof; and

(c) an isolated β₂ integrin or a functional variant thereof;

a test kit suitable for use in identifying a test substance which is capable of inhibiting the interaction between a streptococcal M protein or a functional variant thereof, fibrinogen or a functional variant thereof and PMNs, which kit comprises:

(a) a streptococcal M protein or a functional variant thereof;

(b) fibrinogen or a functional variant thereof; and

(c) one or more PMNs;

an anti-streptococcal agent identified by a method of the invention;

an anti-streptococcal agent identified by a method of the invention for use in a method of treatment of the human or animal body by therapy;

use of an integrin antagonist in the manufacture of a medicament for the treatment of a streptococcal infection;

use of an inhibitor of the interaction between streptococcal M protein, fibrinogen and β₂ integrin in the manufacture of a medicament for the treatment of a streptococcal infection;

use of an agent identified by a method of the invention in the manufacture of a medicament for the treatment of a streptococcal infection;

a method of treating an individual suffering from a streptococcal infection comprising administering a therapeutically effective amount of an agent identified by a method of the invention to a said individual;

a method of treating an individual suffering from a streptococcal infection comprising administering a therapeutically effective amount of an integrin antagonist to a said individual;

a method of treating an individual suffering from a streptococcal infection comprising administering a therapeutically effective amount of an inhibitor of the interaction between streptococcal M protein, fibrinogen and β₂ integrin to a said individual;

a pharmaceutical composition comprising an inhibitor of the interaction between streptococcal M protein, fibrinogen and β₂ integrin identified by a method of the invention and a pharmaceutically acceptable carrier or diluent;

a method for providing a pharmaceutical composition, which method comprises:

(a) identifying an agent that inhibits the interaction between streptococcal M protein, fibrinogen and β₂ integrin by a method of the invention; and

(b) formulating the inhibitor thus identified with a pharmaceutically acceptable carrier or diluent;

a method of treating an individual suffering from a streptococcal infection, which method comprises:

(c) identifying an agent that inhibits the interaction between streptococcal M protein, fibrinogen and β₂ integrin by a method of the invention; and

(d) administering a therapeutically effective amount of the inhibitor thus identified to a said individual.

### BRIEF DESCRIPTION OF THE DRAWINGS

FIG. 1 shows the release of HBP in human blood. Panel A: Human blood was incubated with M1 protein, protein H, SpeB, protein SIC, fMLP, lipoteichoic acid (LTA), or hyaloronic acid (HA) for 30 min at 37° C. Cells were pelleted and the concentration of HBP in the supernatants was determined by ELISA. The total amount of HBP in blood was determined by lysing cells with Triton X-100, and the amount of HBP released after incubation without stimulation for 30 min at 37° C. was considered as background. The figure presents the mean±SD of three independently performed experiments, each done in duplicate. Panel B: Human blood was stimulated with M1 protein, M1 protein fragments A-S and S-C3 (schematically depicted at the top), or protein H for 30 min at 37° C. Cells were pelleted and the concentration of HBP in the supematants was determined by ELISA. The figure presents the mean±SD of three independently performed experiments, each done in duplicate. Panel C: Serial dilutions of supernatants from overnight cultures of strains AP1 and MC25, or growth medium alone were added to human blood and the release of HBP was determined.

FIG. 2 shows the inhibition of M1 protein-induced release of HBP in human blood. Human blood was incubated with tBoc (100 μM), pertussis toxin (1 μg/ml), genistein (100 μM), wortmannin (0,2 μM), BAPTAM/EGTA (10 μM/1 mM), EGTA (1 mM), AG1478 (2 μM), GF109203 (2 μM), H-89 (1 μM), PD98059 (20 μM), or U-73122 (10 μM) in the presence or absence of M1 protein (1 μg/ml) for 30 min at 37° C. Cells were centrifuged and the concentration of HBP in the supernatants was determined by ELISA. The results are expressed as percent of released HBP in the presence of inhibitor relative to release of HBP in the absence of inhibitor (100%). The figure presents the mean±SD of three independently performed experiments, each done in duplicate.

FIG. 3 shows that M1 protein-induced release of HBP correlates with M1 protein-induced precipitation of plasma proteins. Panel A: Samples of 10% human plasma in PBS (1 ml) were incubated with ¹²⁵I-M1 protein (10⁵ cpm/ml, approximately 1 ng) in the presence (0.01 μ/ml, 0.1 μg/ml, 0.2 μg/ml, 1 μg/ml, and 10 μ/ml) or absence of non-labeled M1 protein for 30 min at 37° C. Samples were centrifuged and the radioactivity of the pellets was measured. Results are presented as percentage of added total radioactivity and the figure shows the mean±SD of three independent experiments, each done in duplicate. Panel B: Human whole blood was treated with M1 protein (0.01 μg/ml, 0.1 μg/ml, 0.2 μg/ml, 1 μg/ml, or 10 μg/ml) for 30 min at 37° C. Cells were centrifuged and the amount of HBP in the supernatants was determined. Panel C: One ml samples of human plasma (10% in PBS) or fibrinogen (300 μg/ml in PBS) were incubated with ¹²⁵I-M1 protein (10⁵ cpm/ml, approximately 1 ng) in the absence or presence of non-labeled M1 protein (0.01 μg/ml, 0.1 μg/ml, 0.2 μg/ml, 1 μg/ml, or 10 μg/ml). After 30 min of incubation at 37° C., samples were centrifuged and the radioactivity of the pellets was measured. Results are presented as percentage of total radioactivity. The figure presents the mean±SD of three independent experiments, each done in duplicate.

FIG. 4 shows that M1 protein-induced precipitates formed in a fibrinogen solution or in plasma cause HBP release. M1 protein (1 μg/ml) was added to 10% human plasma or fibrinogen (300 μg/ml) in PBS for 30 min. After a centrifugation step, the resulting pellets were resuspended and incubated with 10% human blood diluted in PBS for 30 min followed by the measurement of released HBP. Plasma or fibrinogen solutions devoid of M1 protein were treated in the same way and served as negative controls. The figure presents the mean±SD of four independently performed experiments.

FIG. 5 shows inhibition of the M1 protein-induced HBP release by fibrinogen derived peptides and antibodies to CD18. Panel A: Human plasma was incubated with peptides Gly-Pro-Arg-Pro, Gly-His-Arg-Pro (100 μg/ml), or buffer alone for 15 min at 37° C. Clotting was initiated by the addition of thrombin and the clotting time was determined. Panel B: M1 protein was added to whole human blood (1 μg/ml) followed by the addition of different amounts of Gly-Pro-Arg-Pro, Gly-His-Arg-Pro, antibody mAB IB4 to CD18, or antibody AS88 (directed against human H-kininogen). After 30 min of incubation at 37° C., cells were centrifuged and the amount of HBP in the supernatants was determined. Data are expressed as percent of HBP release induced by M1 protein alone, and the bars represent means±SD of 3 experiments, each done in duplicate.

### BRIEF DESCRIPTION OF THE SEQUENCE LISTING

SEQ ID NO: 1 shows the amino acid sequence of the M1 protein of Streptococcus pyogenes (NCBI Accession Number NP_{—}269973).

SEQ ID NO: 2 shows the amino acid sequence of a peptide derived from the NH₂-terminal region of fibrinogen.

SEQ ID NO: 3 shows the amino acid sequence of a second peptide derived from the NH₂-terminal region of fibrinogen.

SEQ ID NO: 4 is a RT-PCR primer used in the Example.

SEQ ID NO: 5 shows the amino acid sequence of the human fibrinogen α chain isoform α preproprotein (NCBI Accession Number NP_{—}068657).

SEQ ID NO: 6 shows the amino acid sequence of the human fibrinogen β chain precursor (NCBI Accession Number P02675).

SEQ ID NO: 7 shows the amino acid sequence of the human fibrinogen γ chain isoform γ-B precursor (NCBI Accession Number NP_{—}068656).

SEQ ID NO: 8 shows the amino acid sequence of human integrin α_{M} chain precursor (NCBI Accession Number NP_{—}000623).

SEQ ID NO: 9 shows the amino acid sequence of human integrin a subunit (αₓ chain) precursor (NCBI Accession Number AAA51620).

SEQ ID NO: 10 shows the amino acid sequence of human β₂ integrin chain precursor (NCBI Accession Number NP_{—}000202).

### DETAILED DESCRIPTION OF THE INVENTION

The invention provides methods for identifying an anti-streptococcal agent. A suitable method of the invention consists essentially of:

contacting (i) an isolated streptococcal M protein or a functional variant thereof, (ii) isolated fibrinogen or a functional variant thereof, and (iii) an isolated β₂ integrin or a functional variant thereof with a test substance under conditions that would permit the components to interact in the absence of the test substance; and

determining whether the test substance is capable of inhibiting the interaction between the components.

It can then be readily determined whether the test substance is an anti-streptococcal agent.

An isolated streptococcal M protein or a functional variant thereof is provided as a first component. Streptococcal M proteins and M-like proteins are well known. There are more than 80 different streptococcal M proteins. The M protein of the invention may be, for instance, M1, M3, M11, M12 or M28. The M protein is preferably M1 or M3. Typically, the M protein is derived from S. pyogenes. Preferably, the M protein is M1 protein of S. pyogenes. The amino acid sequence of the M1 protein of S. pyogenes is set out in SEQ ID NO: 1.

A functional variant of a streptococcal M protein maintains the ability to form a complex with fibrinogen. Such a complex is capable of binding to a β₂ integrin. The functional variant may be a fragment of a streptococcal M protein. A functional variant of a streptococcal M protein typically binds specifically to fibrinogen. Binding of M proteins to fibrinogen may be analysed as described by Åkesson et al. (Åkesson et al., 1994, Biochem. J., 300, 877-886). The affinity constant for the interaction between a functional variant of a streptococcal M protein and fibrinogen is typically from 1×10⁻⁶M to 1×10⁻¹²M. For example, the affinity constant may be from 1×10⁻⁷M to 1×10⁻¹¹M or from 1×10⁻⁸M to 1×10⁻¹⁰M.

Typically, the binding affinity for fibrinogen of such a functional variant is substantially the same as that of the wild type M protein. Alternatively, the binding affinity for fibrinogen may be greater or less than that of the wild type streptococcal M protein. For example, a functional variant may have a binding affinity for fibrinogen which is at least 95%, at least 90%, at least 85%, at least 80%, at least 75%, or at least 70% of that of the wild type streptococcal M protein. Alternatively, the binding affinity for fibrinogen of the functional variant may be at least 105%, at least 110%, at least 120%, or at least 130% of that of the wild type streptococcal M protein. For instance, the binding affinity for fibrinogen of a functional variant of a streptococcal M protein may be from 95% to 105%, from 90% to 110%, from 85% to 120%, from 80% to 130%, from 75% to 140% or from 70% to 150% of that of the wild type. In each case, the affinity constant for the interaction between a functional variant of a streptococcal M protein and fibrinogen is typically from 1×10⁻⁶M to 1×10⁻¹²M. For example, the affinity constant may be from 1×10⁻⁷M to 1×10⁻¹¹M or from 1×10⁻⁸M to 1×10⁻¹⁰M.

A functional variant of a streptococcal M protein may be a polypeptide which has a sequence similar to that of an M protein such as the wild type M1 protein of S. pyogenes of SEQ ID NO: 1. Thus a functional variant will generally have at least 60%, at least 70%, at least 80%, at least 90%, at least 95%, at least 98% or at least 99% sequence identity to that of the streptococcal M protein calculated over the full length of those sequences. The UWGCG Package provides the BESTFIT program which can be used to calculate identity (for example used on its default settings) (Devereux et al (1984) Nucleic Acids Research 12, 387-395). The PILEUP and BLAST algorithms can alternatively be used to calculate identity or line up sequences (typically on their default settings), for example as described in Altschul S. F. (1993) J Mol Evol 36:290-300; Altschul, S. F. et al (1990) J Mol Biol 215:403-10. Identity may Therefore be calculated using the UWGCG package, using the BESTFIT program on its default settings. Alternatively, sequence identity can be calculated using the PILEUP or BLAST algorithms. BLAST may be used on its default settings.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff(1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

A functional variant may be a modified version of a streptococcal M protein such as the S. pyogenes M1 protein with the amino acid sequence of SEQ ID NO: 1. The sequence of the modified version is different to that of the wild type M protein. The modified version of a wild type M protein may have, for example, amino acid substitutions, deletions or additions. At least 1, at least 2, at least 3, at least 5, at least 10 or at least 20 amino acid substitutions or deletions, for example, may be made, up to a maximum of 100 or 50 or 30. For example, from 1 to 100, from 2 to 50, from 3 to 30, or from 5 to 15 amino acid substitutions or deletions may be made. Typically, if substitutions are made, the substitutions will be conservative substitutions, for example according to the following Table. Amino acids in the same block in the second column and preferably in the same line in the third column may be substituted for each other. Deletions are preferably deletions of amino acids from one or both ends of the sequence of the streptococcal M protein. Alternatively, deletions are of regions not involved in the interaction with fibrinogen. For example, the deletion may be in the S-C3 fragment of S. pyogenes M1 protein.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The streptococcal M protein or a functional variant thereof may be fused to an additional heterologous polypeptide sequence to produce a fusion polypeptide. Thus, additional amino acid residues may be provided at, for example, one or both termnini of the streptococcal M protein or a functional variant thereof. The additional sequence may perform any known function. Typically, it may be added for the purpose of providing a carrier polypeptide, by which the streptococcal M protein or functional variant thereof can be, for example, affixed to a label, solid matrix or carrier. Thus the first component for use in the invention may be in the form of a fusion polypeptide which comprises heterologous sequences. Indeed, in practice it may often be convenient to use fusion polypeptides. This is because fusion polypeptides may be easily and cheaply produced in recombinant cell lines, for example recombinant bacterial or insect cell lines. Fusion polypeptides may be expressed at higher levels than the wild-type streptococcal M protein or functional variant thereof. Typically this is due to increased translation of the encoding RNA or decreased degradation. In addition, fusion polypeptides may be easy to identify and isolate. Typically, fusion polypeptides will comprise a polypeptide sequence as described above and a carrier or linker sequence. The carrier or linker sequence will typically be derived from a non-human, preferably a non-mammalian source, for example a bacterial source. This is to minimize the occurrence of non-specific interactions between heterologous sequences in the fusion polypeptide and fibrinogen, which is the target of the structural M protein or functional variant thereof.

The streptococcal M protein or a functional variant thereof may be modified by, for example, addition of histidine residues, a T7 tag or glutathione S-transferase, to assist in its isolation. Alternatively, the heterologous sequence may, for example, promote secretion of the streptococcal M protein or functional variant thereof from a cell or target its expression to a particular subcellular location, such as the cell membrane. Amino acid carriers can be from 1 to 400 amino acids in length or more typically from 5 to 200 residues in length. The M protein or functional variant thereof may be linked to a carrier polypeptide directly or via an intervening linker sequence. Typical amino acid residues used for linking are tyrosine, cysteine, lysine, glutamic acid or aspartic acid.

Streptococcal M proteins or functional variants thereof may be chemically modified, for example, post-translationally modified. For example they may comprise modified amino acid residues or may be glycosylated. They can be in a variety of forms of polypeptide derivatives, including amides and conjugates with polypeptides.

Chemically modified streptococcal M proteins or functional variants thereof also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

Also included as chemically modified streptococcal M proteins or functional variants thereof are those which contain one or more naturally occurring amino acid derivatives of the twenty standard amino acids. For example, 4-hydroxyproline may be substituted for proline or homoserine may be substituted for serine.

A streptococcal M protein or a functional variant thereof and/or other polypeptides used as part of a first component may carry a revealing label. Suitable labels include radioisotopes such as ¹²⁵I, ³²p or ³⁵S, fluorescent labels, enzyme labels, or other protein labels such as biotin.

The second component comprises isolated fibrinogen or a functional variant thereof. Fibrinogen is a soluble plasma protein which is converted to insoluble fibrin in the blood by the action of the enzyme thrombin. This contributes to the formation of a blood clot. Fibrinogen is composed of six peptide chains. These are arranged in two identical subunits, each composed of an Aα, a Bβ and a γ chain, joined by disulphide bonds. Streptococcal M protein binds to fibrinogen (Kantor, 1965, J. Exp. Med., 121, 849-859) with high affinity (Åkesson et al., 1994, Biochem. J., 300, 877-886; Berge et al., 1997, J. Biol. Chem., 272, 20774-20781). Fibrinogen also binds to PMNs via β₂ integrins (Altieri, 1999, Thromb. Haemost., 82, 781-786). The binding site for the β₂ integrin Mac1 has been mapped to the N-terminal region of the Aα chain of fibrinogen. In addition, the unique sequence KQAGDV, which is found at the C-terminal end of the γ chain, is essential for integrin binding.

A functional variant of fibrinogen maintains the ability to bind to and thus form a complex with a streptococcal M protein. Such a complex is then capable of binding to a β₂ integrin. The functional variant of fibrinogen typically shows substantially specific binding to a streptococcal M protein. The affinity constant for the interaction between a functional variant of fibrinogen and a streptococcal M protein is typically from 1×10⁻⁶M to 1×10⁻¹²M. For example, the affinity constant may be from 1×10⁻⁷M to 1×10⁻¹¹M or from 1×10⁻⁸M to 1×10⁻¹⁰M.

Typically, the binding affinity of a functional variant of fibrinogen for a streptococcal M protein is substantially the same as that of wild type fibrinogen.

Alternatively, the binding affinity for the streptococcal M protein may be greater or less than that of wild type fibrinogen. For example, a functional variant of fibrinogen may to have a binding affinity for streptococcal M protein which is at least 95%, at least 90%, at least 85%, at least 80%, at least 75% or at least 70% of that of wild type fibrinogen.

Alternatively, the binding affinity for the streptococcal M protein of the functional variant may be at least 105%, at least 110%, at least 120% or at least 130% of that of wild type fibrinogen. For example, the binding affinity for streptococcal M protein of the functional variant may be from 95% to 105%, from 90% to 110%, from 85% to 120%, from 80% to 130%, from 75% to 140% or from 70% to 150% of that of wild type fibrinogen. In each case, typically the affinity constant for the interaction between a functional variant of fibrinogen and a streptococcal M protein is typically from 1×10⁻⁶M to 1×10⁻¹²M. For example, the affinity constant may be from 1×10⁻⁷M to 1×10⁻¹¹M or from 1×10⁻¹²M to 1×10⁻¹⁰M.

A functional variant of fibrinogen may contain an Aα chain which has a sequence similar to that of the native Aα chain of fibrinogen, such as the human Aα chain shown in SEQ ID NO: 5. A functional variant of fibrinogen may contain a Bβ chain which has a sequence similar to that of the native Bβ chain, for example the human Bβ chain shown in SEQ ID NO: 6. A functional variant of fibrinogen may contain a γ chain whose sequence is similar to that of the native γ chain such as the human γ chain of SEQ ID NO: 7. An Aα, Bβ or γ chain can therefore have at least 60%, at least 70%, at least 80%, at least 90%, at least 95%, at least 98% or at least 99% sequence identity to that of the native Aα, Bβ or γ chain of fibrinogen, such as the human Aα, Bβ or γ chains shown in SEQ ID NOs 5 to 7, calculated over the full length of those sequences. However, the chains must still be capable of assembly into a functional molecule. Sequence identity can be calculated using the methods described above. The BESTFIT program of the UWGCG package may be used on its default settings. Alternatively the PILEUP or BLAST algorithms may be used on their default settings.

A functional variant may be a modified version of fibrinogen which may have, for example, amino acid substitutions, deletions or additions in the Aα and/or the Bβ and/or the γ chains of fibrinogen. Such substitutions, deletions or additions may be made, for example, to the sequences of the human Aα, Bβ or γ chains shown in SEQ ID NOs 5 to 7. Any combination of chains or all of the chains may be modified. However, any deletions, additions or substitutions must still allow the Aα, Bβ and γ chains of fibrinogen to assemble into a functional molecule. At least 1, at least 2, at least 3, at least 5, at least 10, at least 20 or at least 50 amino acid substitutions or deletions, for example, may be made up to a maximum of 70 or 50 or 30 in each chain. For example, from 1 to 70, from 2 to 50, from 3 to 30 or from 5 to 20 amino acid substitutions or deletions may be made. Typically, if substitutions are made, the substitutions will be conservative substitutions as described above. Deletions are preferably deletions of amino acids from one or both ends of the sequence of the Aα, Bβ or γ chains of fibrinogen such as those shown in SEQ ID NOs 5 to 7. Alternatively, deletions are of regions not involved with the interaction with streptococcal M proteins.

Any of the polypeptide chains of fibrinogen or a functional variant thereof may be fused to an additional heterologous polypeptide sequence to produce a fusion polypeptide, as long as the polypeptide chains are still capable of assembling into a functional molecule. Such a fusion polypeptide may be a carrier polypeptide or contain a linker sequence. Such polypeptides are described above.

The polypeptide chains of fibrinogen or a functional variant thereof may be chemically modified as described above. Alternatively the polypeptide chains of fibrinogen or a functional variant thereof may carry a revealing label. Suitable labels are described above.

The third component comprises an isolated β₂ integrin or a functional variant thereof. Integrins are a large family of heterodimeric cell surface adhesion receptors, composed of a β chain and an α chain. Each subunit is composed of a large extracellular domain, a single transmembrane domain and a short cytoplasmic domain. A number of α and β subunits have been identified and these can associate in a restricted manner. An α subunit usually only associates with a particular β subunit but β subunits are more promiscuous. β₂ integrins are the most abundant integrins expressed by PMNs. Four different β chains (α_{M}, α_{L}, α_{X} and α_{D}) can associate with the β₂ chain. Of these, α_{M}β_{2,} also known as CD11 b/CD18, and α_{X}β₂, also known as CD11 c/CD18, are the main integrins expressed on PMNs. These are the receptors for fibrinogen.

A functional variant of a β₂ integrin maintains the ability to bind to a streptococcal M protein-fibrinogen complex. A functional variant of a β₂ integrin typically binds specifically to streptococcal M protein-fibrinogen complex. The affinity constant for the interaction between a functional variant of a β₂ integrin and streptococcal M protein-fibrinogen complex is typically from 1×10⁻⁶M to 1×10⁻¹²M. For example, the affinity constant may be from 1×10⁻⁷M to 1×10⁻¹¹M or from 1×10⁻⁸M to 1×10⁻¹⁰M.

Typically, the binding affinity of a functional variant of a β₂ integrin for a streptococcal M protein-fibrinogen complex is substantially the same as that of the wild type β₂ integrin. Alternatively, the binding affinity for streptococcal M protein-fibrinogen complexes may be greater or less than that of the wild type β₂ integrin. For example, the binding affinity of the functional variant of the β₂ integrin for streptococcal M protein-fibrinogen complexes may be at least 95%, at least 90%, at least 85%, at least 80%, at least 75% or at least 70% of that of the wild type β₂ integrin. Alternatively, the binding affinity of the functional variant may be at least 110%, at least 120%, or at least 130% of that of the wild type β₂ integrin. For instance, the binding affinity for streptococcal M protein-fibrinogen complexes of the functional variant may be from 70% to 160%, from 75% to 150%, from 80% to 140%, from 85% to 130%, from 90% to 120% or from 95% to 110% of that of the wild type β₂ integrin. In each case, typically the affinity constant for the interaction between a functional variant of a β₂ integrin and streptococcal M protein-fibrinogen complex is typically from 1×10⁻⁶ M to 1×10⁻¹²M. For example, the affinity constant may be from 1×10⁻⁷M to 1×10⁻¹¹M or from 1×10⁻⁸M to 1×10⁻¹⁰M.

A functional variant of a β₂ integrin may contain an α and/or a β₂ chain which has a sequence similar to that of either the native α or the native β₂ chain of a β₂ integrin. For example, the α chain may have a sequence similar to that of the human α_{M} chain shown in SEQ ID NO: 8 or to that of the human α_{X} chain shown in SEQ ID NO: 9. The β₂ chain may have a sequence similar to that of the human β₂ chain shown in SEQ D NO: 10. Thus an α and/or a β₂ chain can therefore have at least 60%, at least 70%, at least 80%, at least 85%, at least 90%, at least 95%, at least 98% or at least 99% sequence identity to that of the native a or β₂ chain, such as those of SEQ ID NOs 8 to 10, calculated over the full length of those sequences. Again, sequence identity can be calculated using any of the packages described above. The BESTFIT program of the UWGCG package may be used on its default settings. Alternatively, the PILEUP or BLAST algorithms may be used on their default settings.

A functional variant of a β₂ integrin may be a modified version of a β₂ integrin which has, for example, amino acid substitutions, deletions or additions in either or both of the a and β₂ chains. For example, the α_{M}, α_{X} or β₂ chains may contain substitutions, deletions or additions to the sequence of the native α_{M}, α_{X} or β₂ chain such as those of the human α_{M}, αₓ and β₂ chains shown in SEQ ID NOs 8 to 10. At least 1, at least 2, at least 5, at least 10, at least 30, at least 50 or at least 100 amino acid substitutions or deletions, for example, may be made, up to a maximum of 200, 100, 50 or 30 in either or both of the α and β₂ chains. For example, from 1 to 200, from 2 to 150, from 3 to 100, from 5 to 50 or from 10 to 30 amino acid substitutions or deletions may be made. Typically, any substitutions will be conservative substitutions as described above. Deletions are preferably deletions of amino acids from one or both ends of the sequence of the α or β₂ chain such as any of the sequences of SEQ ID NOs 8 to 10. Alternatively, deletions are of regions not involved in the interaction with streptococcal M protein-fibrinogen complexes.

The α or β₂ chain of a β₂ integrin or a functional variant thereof may be fused to a heterologous polypeptide sequence to produce a fusion polypeptide. This may produce a carrier polypeptide, as described above. Alternatively, the α or β₂ chain of a β₂ integrin or functional variant thereof may be modified by, for example, addition of amino acid residues to assist in its isolation. It may be linked to a carrier polypeptide directly or via a linker sequence. The α or β₂ chain of a β₂ integrin or functional variant thereof may be chemically modified as described above, or it may be carry a revealing label. Suitable labels are described above.

The method of the invention can be carried out according to any suitable protocol. Preferably, the method is adapted so that it can be carried out in a single reaction vessel such as a single well of a plastic microtiter plate and thus can be adapted for high throughput screening. Preferably, therefore, the assay is an in vitro assay.

A streptococcal M protein or a functional variant thereof and/or other polypeptides used as part of a first component may be expressed using recombinant DNA techniques. For example, suitable polypeptides may be expressed in, for example, bacterial or insect cell lines (see, for example, Munger et al., 1998, Molecular Biology of the Cell, 9, 2627-2638). Typically, a recombinant streptococcal M protein can be produced by expression in E. coli. The M protein is preferably S. pyogenes M1 protein. Recombinant polypeptides are produced by providing a polynucleotide encoding a streptococcal M protein or functional variant thereof. Such polynucleotides are provided with suitable control elements, such as promoter sequences, and provided in expression vectors and the like for expression of streptococcal M protein or a functional variant thereof. Suitable polypeptides may be isolated biochemically from any suitable bacteria.

Alternatively, M protein can be obtained from streptococcal cells that express M proteins endogenously or through the use of recombinant techniques. For example, an M protein from S. pyogenes may be produced by treating S. pyogenes cells with a protease. The M protein is preferably M1 protein. The protease may be endogenous to S. pyogenes, for example the S. pyogenes cysteine proteinase SpeB. Alternatively, the protease may be derived from PMNs. Typically, the PMN protease is produced by lysing PMNs. A protease may also be produced recombinantly. M protein may alternatively be obtained by expression of a truncated version of the M protein which lacks the membrane spanning region (Collin and Olsén, 2000, Mol. Microbiol., 36, 1306-1318). Such a protein may be expressed in S. pyogenes or E.coli and will be secreted by the bacteria without the need for proteolytic cleavage.

Alternatively, a streptococcal M protein or a functional variant thereof may be chemically synthesized. Synthetic techniques, such as a solid-phase Merrifield-type synthesis, may be preferred for reasons of purity, antigenic specificity, freedom from unwanted side products and ease of production. Suitable techniques for solid-phase peptide synthesis are well known to those skilled in the art (see for example, Merrifield et al., 1969, Adv. Enzymol 32, 221-96 and Fields et al., 1990, Int. J. Peptide Protein Res, 35, 161-214). In general, solid-phase synthesis methods comprise the sequential addition of one or more amino acid residues or suitably protected amino acid residues to a growing peptide chain.

Fibrinogen or a functional variant thereof may be produced by recombinant methods such as expression in bacterial or insect cell lines as described above. Alternatively, fibrinogen or a functional variant thereof may be chemically synthesized. Fibrinogen may be isolated from human blood, preferably from human plasma.

The streptococcal M protein or a functional variant thereof may be provided in association with fibrinogen or a functional variant thereof. That is to say, a complex of streptococcal M protein or a functional variant thereof and fibrinogen or a functional variant thereof can be used in the invention. Such a complex will be capable of binding to β₂ integrins. Alternatively, the streptococcal M protein or functional variant thereof and fibrinogen or functional thereof may be provided separately.

A β₂ integrin or a functional variant thereof may be produced by recombinant methods or be chemically synthesized as described above. The β₂ integrin may be isolated from PMN lysate.

The streptococcal M protein, fibrinogen and β₂ integrin used in the method described above are provided in substantially isolated form. That is to say that the streptococcal M protein, fibrinogen and β₂ integrin or functional variant of any of these may be produced as described above and then isolated. They will generally comprise at least 80%, for instance at least 90%, 95% or 99% by weight of the dry mass in the preparation.

Streptococcal M protein and/or fibrinogen and/or β₂ integrin used in the invention may be present in non-naturally occurring form. The streptococcal M protein and/or fibrinogen and/or β₂ integrin may be in substantially purified form.

An alternative method of the invention consists essentially of:

contacting (i) a streptococcal M protein or a functional variant thereof, (ii) fibrinogen or a functional variant thereof, and (iii) one or more polymorphonuclear neutrophils (PMNs) with a test substance under conditions that would permit the components to interact in the absence of the test substance; and

monitoring any inhibition of the activation of PMNs.

It can there be readily determined whether the test substance is an anti-streptococcal agent.

The first component, streptococcal M protein or functional variant thereof, and the second component, fibrinogen or a functional variant thereof, may be provided by any of the methods described above. The PMNs may be provided in human blood. The streptococcal M protein and fibrinogen bind to the PMNs via β₂ integrins on the surface of the PMNs.

In a typical method of the invention, isolated streptococcal M protein, isolated fibrinogen and isolated β₂ integrin are mixed together. A test substance is then added to the mixture under conditions that would permit the components to interact in the absence of the test substance. Suitable conditions can be identified by mixing together the isolated streptococcal M protein, isolated fibrinogen and isolated β₂ integrin in the absence of the test substance to determine whether the components interact in the absence of the test substance, for example by determining whether the components form aggregates in the absence of the test substance. Such aggregates can be detected by electron microscopy. Alternatively, radiolabelled proteins can be used to spike the reaction mixture and the amount of radioactivity in the aggregates can be used to quantify the formation of aggregates.

In an alternative method of the invention, PMNs are reconstituted with a mixture of streptococcal M protein and plasma (to provide fibrinogen). A test substance is then added to the mixture under conditions that would permit the components to interact in the absence of the test substance. Suitable conditions can be identified by reconstituting the PMNs with a mixture of streptococcal M protein and plasma in the absence of the test substance and determining whether the components form aggregates or whether the PMNs are activated in the absence of the test substance. The activation of PMNs is typically determined by monitoring the release of HBP.

A cell adhesion assay may alternatively be carried out. In a typical cell adhesion assay, streptococcal M protein-fibrinogen complexes formed from isolated M protein and isolated fibrinogen are coated onto the walls of the suitable vessel, in particular the well of a plastic microtiter plate. In one suitable assay format, the third component β₂ integrin, produced, for example, chemically or recombinantly and then isolated is simply added to the assay vessel along with a test substance. Binding of the β₂ integrin to the M protein-fibrinogen complex can be followed by the use of β₂ integrin which carries a label, for example a radioactive label or a fluorescent label.

Alternatively, in another suitable assay format, PMN cells are added to the vessel and allowed to interact with streptococcal M protein-fibrinogen complexes in the presence of a test product. These complexes may be formed simply by mixing streptococcal M protein with fibrinogen. The number of cells which bind to the M protein-fibrinogen complex is then determined. This may be carried out by, for example, staining the cells and then carrying out spectrophotometry. Optionally, the stain may be eluted and the spectrophotometry carried out on the eluted sample.

In an alternative assay of the invention, M protein-fibrinogen complexes are coated on the walls of the suitable vessel and then PMN cells are added to the vessel and allowed to interact with the M protein-fibrinogen complexes in the presence of a test product. Inhibition of binding between the M protein-fibrinogen complexes and PMNs is then detected by monitoring the activation of the PMNs. Typically, this can be done by measuring the release of heparin binding protein (HBP). A preferred method of the present invention comprises providing S. pyogenes, fibrinogen and PMNs with a test substance to test, as in the assay described above, whether the test substance inhibits binding of the M protein-fibrinogen complexes to β₂ integrin on the surface of the PMNs.

Suitable methods of the invention may be carried out in the presence of suitable buffers.

Suitable control experiments may be carried out. For example, assays may be carried out in the absence of a test substance to monitor the interaction between M protein-fibrinogen complexes and isolated β₂ integrin or PMNs.

Suitable test substances which can be tested in the above methods include combinatorial libraries, defined chemical entities, peptide and peptide mimetics, oligonucleotides and natural product libraries, such as display (e.g. phage display libraries) and antibody products. For example, monoclonal and polyclonal antibodies, single chain antibodies, chimeric antibodies, CDR-grafted antibodies and humanized antibodies may be used. The antibody may be an intact immunoglobulin molecule or a fragment thereof such as a Fab, F(ab′)₂ or Fv fragment. Suitable peptides include the peptide with the sequence GPRP. Suitable antibodies include antibodies directed against the B-repeats of S. pyogenes M1 protein, the monoclonal antibody IB4 and antibodies to CD11c.

Suitable test substances also include integrin antagonists, typically β₂ integrin antagonists. Suitable integrin antagonists include anti-integrin antibodies, peptide mimetics and non-peptide mimetics. Anti-integrin antibodies may be of any of the types of antibodies described above. Antagonists can be identified by testing whether they inhibit the action of an agonist which, in the absence of the antagonist, would otherwise bind to the receptor and exert a biological effect.

Typically, organic molecules will be screened, preferably small organic molecules which have a molecular weight of from 50 to 2500 daltons. Candidate products can be biomolecules including saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition tested individually. Test substances may be used at a concentration of from 1 nM to 1000 μM, preferably from 1 μM to 100 μM, more preferably from 1 M to 10 μM.

An inhibitor of the interaction between streptococcal M protein, fibrinogen and β₂ integrin is one which produces a measurable reduction in such an interaction in a method described above. An inhibitor of the interaction is one which causes the degree of interaction to be reduced or substantially eliminated, as compared to the degree of interaction in the absence of that inhibitor. Preferred inhibitors are those which inhibit the interaction by at least 10%, at least 20%, at least 30%, at least 40%, at least 50%, at least 60%, at least 70%, at least 80%, at least 90%, at least 95% or at least 99% at a concentration of the inhibitor of 1 μgml⁻¹, 10 μgml⁻¹, 100 μgml⁻¹ , 500 μgml⁻¹, 1 mgml⁻¹, 10 mgml⁻¹, 100 mg ml⁻¹. The percentage inhibition represents the percentage decrease in any interaction between streptococcal M protein, fibrinogen and β₂ integrin in a comparison of assays in the presence and absence of the test substance. Any combination of the above mentioned degrees of percentage inhibition and concentration of inhibitor may be used to a define an inhibitor of the invention, with greater inhibition at lower concentrations being preferred. Test substances which show activity in methods of the invention can be tested in in vivo systems, such as an animal disease model. Thus, candidate inhibitors could be tested for their ability to attenuate inflammation and/or lung lesions caused by streptococci in mice. Thus it can be determined whether test substances identified by methods of the invention are effective anti-streptococcal agents.

Inhibitors of the invention may be in substantially purified form. They may be in substantially isolated form, in which case they will generally comprise at least 80% e.g. at least 90, 95, 97 or 99% by weight of the dry mass in the preparation. The product is typically substantially free of other cellular components. The product may be used in such a substantially isolated, purified or free form in the method of the invention.

The invention also provides test kits. A suitable kit consists essentially of an isolated streptococcal M protein or a functional variant thereof, isolated fibrinogen or a functional variant thereof, and an isolated β₂ integrin or a functional variant thereof. An alternative kit of the invention consists essentially of a streptococcal M protein or a functional variant thereof, fibrinogen or a functional variant thereof, and one or more PMNs. The test kit may also comprise means for determining whether a test substance disrupts the interaction between the components. Such a means may be the reagents and solutions required to determine whether streptococcal M proteins, fibrinogen and β₂ integrin or PMNs interact according to any method known in the art. A test kit of the invention may also comprise one or more buffers. Kits of the invention are optionally provided with packaging and preferably comprise instructions for the use of the kit.

Inhibitors of the invention may be used in a method of treatment of the human or animal body by therapy. In particular, inhibitors of the present invention may be used in the treatment of streptococcal infections, preferably in the treatment of infection by S. pyogenes. Inhibitors can be used to improve the condition of a patient suffering from a streptococcal infection. Such inhibitors may be used in the treatment of humans or animals. Such inhibitors may be used in prophylactic treatment, for example, in immunosuppressed patients more susceptible to streptococcal infection Alternatively, such agents may be used in patients demonstrated to have a streptococcal infection to alleviate the symptoms thereof. A therapeutically effective amount of inhibitor may be given to a host in need thereof.

The inhibitors may be administered in a variety of dosage forms. Thus, they can be administered orally, for example as tablets, troches, lozenges, aqueous or oily suspensions, dispersible powders or granules. They may also be administered parenterally, either subcutaneously, intravenously, intramuscularly, intrasternally, transdermally or by infusion techniques. They may also be administered as suppositories. A physician will be able to determine the required route of administration for each particular patient.

The formulation of an inhibitor for use in preventing or treating streptococcal infection will depend upon factors such as the nature of the exact substance, whether a pharmaceutical or veterinary use is intended, etc. An inhibitor may be formulated for simultaneous, separate or sequential use.

An inhibitor is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose s with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of an inhibitor is administered to an individual in need thereof. The dose of the inhibitor may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific substance, the age, weight and conditions of the subject to be treated, the type and severity of the degeneration and the frequency and route of administration. Preferably, daily dosage levels are from 5 mg to 2 g.

The following Example illustrates the invention:

### EXAMPLE

### Materials and Methods

Reagents. Neutrophil Isolation Medium (NIM) was purchased from Cardinal Associates Inc. (Santa Fe, N.Mex.). RPMI 1640 medium with Glutamax I (trade mark), Minimum Essential Medium (MEM) with Earle's salts and L-glutamine, fetal bovine serum, and penicillin (5000 units/ml)/streptomycin (5000 μ/ml) solution were purchased from Life Technologies (Täby, Sweden). Ionomycin and formyl-methionyl-leucyl-phenylalanine (fMLP) were obtained from Calbiochem (La Jolla, Calif.). The acetoxymethyl ester of N,N′-(1,2-ethanediylbis(oxy-2,1-phenylene))bis(N-(carboxymethyl)) (BAPTA), and ProLonge® Antifade Kit were from Molecular Probes (Eugene, Oreg.). 4-(2-hydroxyethyl)-1-piperazineethanesulphonic acid (HEPES) was from Merck (Whitehouse Station, N.J.). Streptococcal cysteine proteinase (SpeB) zymogen was purified from the medium of AP1 bacteria by ammonium sulfate precipitation (80% w/v) followed by fractionation on S-Sepharose (Berge et al., 1997, J. Biol. Chem., 272, 20774-20781). Recombinant M1 protein, fragments A-S and S-C3, and protein H were obtained by expression in E. coli and purified as described earlier (Åkesson et al., 1994, Biochem. J., 300, 877-886; Berge et al., 1997, J. Biol. Chem., 272, 20774-20781). Recombinant human HBP was produced using the baculovirus expression system in Sf9 insect cells (Invitrogen Corp., Carlsbad, Calif.) and was purified as described (Laemnili, 1970, Nature, 227, 680-685). Lipoteichoic acid (LTA), hyaluronic acid (HA), and bovine serum albumin (BSA) were from Sigma Chemical Co. (St. Louis, Mo.). Mouse mAB 2F23C3 and rabbit antiserum (409A) to recombinant HBP were prepared and purified as described earlier (Lindmark et al., J. Leukoc.,Biol., 66, 634-643) and peroxidase-conjugated goat anti-rabbit IgG was from Bio-Rad Laboratories (Richmond, Calif.). Peptides H-2935 (Gly-Pro-Arg-Pro) and H-2940 (Gly-His-Arg-Pro) were purchased from Bachem Feinchemikalien AG (Bubendorf, Switzerland). Fluanison/fentanyl and midazolam were from Janssen Pharmaceutica, Beers, Belgium and Hoffmnan-La Roche, Basel, Switzerland.

Cell culture, neutrophil isolation, and stimulation of cells. Human PMNs were isolated from fresh heparinized blood of healthy volunteers using NIM, a single step density gradient medium, according to the instructions supplied by the manufacture. PMNs were counted with a hemocytometer, resuspended in MEM medium at 10⁷ cells/ml and maintained on rotation in this medium at room temperature until use. All experiments on isolated PMNs were performed in Na-medium and initiated within 1 h of PMN isolation. Neutrophilic proteinase release was induced by PMN activation through antibody cross-linking of CD11 b/CD18 as described previously (Gautam et al., 2000, J. Exp. Med., 191, 1829-1839).

Bacterial strains. S. pyogenes strain AP1 used in this study is the 40/58 strain from the World Health Organization Collaborating Centre for references and Research on Streptococci, Institute of Hygiene and Epidemiology, Prague, Czech Republic. Its protein binding properties have been described (Åkesson et al., 1990, Immunol., 27, 523-531; Åkesson et al., 1994, Biochem. J., 300, 877-886; Gomi et al., 1990, J. Immunol., v. 144, p. 4046-405²). The MC25 strain, an AP1 mutant strain, devoid of surface-associated M1 protein, was generated as described earlier (Collin and Olsén, 2000, Mol. Microbiol., 36, 1306-1318).

Enzymatic treatment of S. pyogenes. S. pyogenes bacteria (strain AP1) were grown in Todd-Hewitt broth (Difco, Detroit, Mich.) at 37° C. for 16 h and harvested by centrifugation at 3000×g for 20 min. The bacteria were washed twice in PBS and resuspended in PBS to 2×10⁹ cells/ml). Various amounts of secretion products from PMNs were added to bacterial suspensions followed by incubation for 2 h at 37° C. Bacteria were spun down at 3000×g for 20 min, and the resulting pellets and supernatants were saved. Digestions were terminated by addition of SDS sample buffer reducing conditions.

SDS-polyacrylamide gel electrophoresis, Western blotting, and immunoprinting. Proteins were separated by 12.5% (w/v) polyacrylamide gel electrophoresis in the presence of 1% (w/v) SDS (Laemmli, 1970, Nature, 227, 680-685). Molecular weight markers were from Sigma Chemical Co. (St. Louis, Mo.). The resolved proteins were visualized by the silver stain technique. Proteins were also transferred onto nitrocellulose membranes for 30 min at 100 mA (Khyse-Andersen, 1984, J. Biochem. Biophys. Methods, 10, 203-209). The membranes were blocked with PBS containing 5% (w/v) dry milk powder and 0.05% (w/v) Tween-20, pH 7.4. Immunoprinting of the transferred proteins was done according to Towbin et al. (Towbin et al., 1979, Proc. Natl. Acad. Sci. USA, 76, 4350-4354). Polyclonal antibodies against M1 protein, diluted 1:50000 in the blocking buffer, was used. Bound antibodies were detected using a peroxidase-conjugated secondary antibodies against rabbit IgG (dilution 1:3000) followed by a chemiluminescence detection method. Alternatively, membranes were blocked, incubated with fibrinogen (2 μg/ml) followed by immunodetection with antibodies to fibrinogen (1:1000) and peroxidase-conjugated secondary antibodies against rabbit immunoglobulin (1:3000 diluted).

HBP release. 100 μl human blood were diluted in PBS to a final volume of 1.0 ml and incubated with various PMNs-activating components for 30 min at 37° C. Cells were centrifuged (300×g for 15 min) and the supernatant was analyzed by sandwich ELISA. In order to quantify the total amount of HBP in blood, cells were lysed with 0.02% (v/v) Triton X-100, and pelleted as described above.

Determination of HBP. The concentration of HBP in neutrophilic exudates was determined by a sandwich ELISA (Tapper et al., 2002, Blood, 99, 1785-1793). The ELISA was found to be highly specific showing no crossreactivity with elastase, cathepsin G, or proteinase 3.

Precipitation assay. Radiolabeled M1 protein (¹²⁵I-M1 protein). 10,000 cpm was incubated for 30 min with various amounts of non-radiolabeled M1 protein in PBS containing 10% plasma or 0.3 mg/ml fibrinogen. After centrifugation the pellets were resuspended in PBS and the precipitated M protein was detected by γ-counting.

Scanning electron microscopy—Probes were gently applied to Millipore filters (Waters Corporation, Milford). Samples were then sucked down to the filters by a wet filter paper lying underneath. The filters were fixed in 2% (v/v) glutaraldehyde, 0.1 M sodium cacodylate, 0.1 M sucrose, pH 7.2 for 1 h at 4° C., and washed with 0.15 M cacodylate, pH 7.2. The filters were postfixed with 1% (w/v) osmium tetroxide, 0.15 M sodium cacodylate, pH 7.2, for 1 h at 4° C., washed, and stored in cacodylate buffer. Fixed filter paper samples were dehydrated with an ascending ethanol series (10min per step), dried, mounted on aluminum holders, sputtered with palladium/gold, and examined in a Jeol JSM-350 scanning electron microscope.

Thin-sectioning and transmission electron microscopy—Samples were fixed for 1 h at room temperature and then overnight at 4° C. in 2.5% glutaraldehyde in 0.15 M sodium cacodylate, pH 7.4 (cacodylate buffer). Afterwards, they were washed with cacodylate buffer and postfixed for 1 h at room temperature in 1% osmium tetroxide in cacodylate buffer and dehydrated in a graded series of ethanol and then embedded in Epon 812 using acetone as intermediate solvent. Specimens were sectioned with a diamond knife into 50 nm-thick ultrathin sections on an LKB ultramicrotome. The ultrathin sections were stained with uranyl acetate and lead citrate. Specimens were observed in a Jeol JEM 1230 electron microscope operated at 80 kV accelerating voltage. Images were recorded with a Gatan Multiscan 791 CCD camera.

Clotting assay—The thrombin clotting time (TCT) was measured in a coagulometer (Amelung, Lemgo, Germany). Samples of 200 μl human citrate-treated plasma were incubated with 4 μl of peptide H-2395 or H-2940 (5 mg/ml) for 15 min at 37° C. Clotting was initiated by adding 100 μl of the TCT reagent (Sigma Chemicals, St. Louis, Mo.).

Preparation and stimulation of mouse bone marrow cells and leukocytes—For each sample preparation, bone marrow cells and whole blood were collected from 3 to 5 mice. Bone marrow cells were harvested from the femur bones of the mice, pooled and suspended in calcium-free PBS. Whole blood was collected by cardiac puncture and anticoagulated with 10 mM EDTA (Gautam et al., 2001, Nat. Med., 7, 1123-1127). Blood leukocytes were isolated using Dextran sedimentation. Cells from blood and bone marrow were counted using a Burker chamber. The WBC were washed twice in PBS and resuspended to 1×10⁷ cells/ml. In order to stimulate release of granule proteins, WBC (approximately 10⁷ cells/ml) were pre-incubated with cytochalasin B (10 μM) at room temperature for 5 minutes, followed by incubation with 100 nM fMLP for another 30 min at 37° C. After centrifugation (2000×g; 10 min) the supernatant was collected for further analysis. Alternatively, WBC were lysed by adding 1% boiling SDS in 10 mM Tris-HCl pH 7.4. The solution was boiled for an additional 5 min and then sonicated briefly and analyzed by SDS-PAGE, followed by Western blotting and immunoprinting. For functional studies, cells were lysed by incubation in water for 10 minutes followed by a centrifugation step (10 min at 500×g).

RNA preparation—RNA was prepared from bone marrow cells, harvested from murine femur bones. The cells were pelleted by centrifugation at 400 g. Total RNA was then prepared using the Trizol reagent (Gibco Life Technologies) and the purity was assessed from the ratio A_{260/280} (typically>1.8).

RT-PCR—RT-PCR was conducted with GeneAmp/PerkinElmer RNA PCR kit according to the manufacturer's protocol. Briefly, total RNA (500 ng) in water was heated (65° C., 10 min), chilled on ice, and reverse transcribed (20 min, 42° GG GTT GTT GAG AA 3′ derived from the genomic sequence (NM 001700) of human HBP), 1 U/μl RNase inhibitor, and 2.5% de-ionized forrnamide. After denaturation (5 min, 99° C.), samples were amplified in PCR buffer (1.5 mM MgCl₂, 0.2 mM dNTPs, 1 μM primer, 2.5% de-ionized formamide, and 0.05 1 U/μl Taq polymerase) for 20-35 cycles with annealing between 50 and 60° C. and extension at 72° C., using a PerkinElmer/GeneAmp PCR system 2400. Products were analyzed by agarose gel electrophoresis (1% gels).

Animals—Adult male mice (approximately 30 g) of the C57BL/6 strain were used. Animals were anaesthetized with equal parts of fluanison/fentanyl (Hypnorm 10, 0.2 mg/ml) and midazolam (Dormicum, 5 mg/ml) diluted 1:1 with sterile water (dose: 0.2 ml/ mouse i.m.). The anaesthesia was supplemented with inhalation of 2% isoflurane. All animal experiments were approved by the regional ethical committee. Mice were given an intravenous injection of 100 μl of a solution containing 150 μg/ml M1 protein. Alternatively, 100 μl of a solution containing 150 μg/ml M1 protein and 4 mg/ml Gly-Pro-Arg-Pro or Gly-His-Arg-Pro were intravenously injected. As control vehicle alone was applied via the same route. 30 min after injection, mice were sacrificed and the lungs were removed. Alternatively, 100 μl of a bacteria solution (2×10⁹ AP1 bacteria/ml in the presence or absence of 400 μg Gly-Pro-Arg-Pro or Gly-His-Arg-Pro) were injected together with 0,9 ml of air into the dorsal region of the mouse. After 30 min, mice were given an intravenous injection of 100 μl of a solution containing PBS or 2 mg/ml Gly-Pro-Arg-Pro or Gly-His-Arg-Pro, respectively. Six hours after infection, mice were sacrificed and the lungs were removed.

Histochemistry—Mice were sacrificed, lungs rapidly removed by surgery and fixed at 4° C. for 24 h in buffered 4% formalin (pH 7.4; Kebo). Tissues were dehydrated and imbedded in paraffin (Histolab Products AB), cut into 4-μm sections, and mounted. After removal of the paraffin, tissues were stained with Mayers hematoxylin (Histolab Products AB) and eosin (Surgipath Medical Industries, Inc.).

Immunofluorescence and confocal microscopy—Snap-frozen biopsies of tissue, collected either from the epi-center of infection (fascia) or from a distal site with no evidence of inflammation (muscle), from a patient with necrotizing fasciitis caused by an M1T1 S. pyogenes strain (kindly provided by Prof. Donald E Low, Mount Sinai Hospital, Toronto, Canada) were cryosectioned and fixed as previously described (Norrby-Teglund et al., 2001). Tissue sections were initially blocked with 20% fetal calf serum in PBS-saponin (Sigma, St. Louis, Mo.) for 30 minutes followed by avidin and biotin blocking (Vector laboratories, Burlingame, Calif.) 15 minutes each, and finally 30 minutes incubation with PBS-saponin containing 0.1% BSA-c (Aurion, Wageningen, The Netherlands). All antibodies and fluorochromes were diluted in PBS-saponin-BSA-c. Staining for the M1 protein was achieved by incubation with a polyclonal rabbit antiserum against M1 (diluted 1:10000) overnight, followed by a 30 minutes incubation with biotinylated goat-anti-rabbit IgG (diluted 1:500, Vector Laboratories, Burlingame, Calif.), and subsequent addition of streptavidin conjugated Alexa Fluor 488 diluted 1:600 (Molecular Probes, Eugene, Oreg., USA). Double staining for fibrinogen was obtained through direct labelling of purified rabbit anti-fibrinogen antibodies diluted to a concentration of 3 mg/ml (Dakocytomation) by Zenon Alexa fluor 532 IgG labelling kit (Molecular Probes) and incubation with the tissue sections for 90 minutes. Vectashield supplemented with dapi (Vector Lab.) was used as mounting media. A polyclonal rabbit antiserum against the Lancefield group A carbohydrate was used to detect S. pyogenes (Norrby-Teglund et al., 2001) and served as a positive control to verify the specificity of the M1-staining. Single stainings were also performed to assure specificity of staining patterns. For evaluation, the Leica confocal scanner TCS2 AOBS with an inverted Leica DMIRE2 microscope was used.

Results

Neutrophil Proteinases Release M1 Protein from the Surface of S. pyogenes

To test whether M1 protein is released from the streptococcal surface following treatment with human neutrophil proteinases, AP1 bacteria (2×10⁹ bacteria/ml) were incubated with serial dilutions (100 μl, 10 μl or 1 μl) of secretion products (exudates) from PMNs (2×10⁶ cells/ml) stimulated by antibody-crosslinking of CD11b/CD18 for 2 hours at 37° C. Activation of the β₂ integrins by antibody-crosslinking mimics adhesion-dependent receptor engagement and induces the release of neutrophil elastase, cathepsin G, and proteinase 3 (Gautam et al., 2000, J. Exp. Med., 191, 1829-1839), which we confirmed in our experimental settings in an indirect ELISA (data not shown). Incubation of the neutrophil exudates with AP1 bacteria resulted in the solubilization of several streptococcal proteins from the bacterial cell wall. This was seen by centrifugation of bacteria and separating the supernatants by SDS-PAGE (data not shown). The presence of M1 protein among the solubilized proteins was analyzed by Western blot analysis using a polyclonal antiserum against M1 protein. After SDS-PAGE, the solubilized proteins were transferred onto nitrocellulose and probed with antibodies to M1 protein. Bound antibody was detected by a peroxidase-conjugated secondary antibody to rabbit immunoglobulin, followed by the chemiluminescence detection method. The supernatant from untreated bacteria was used as a control.

In the absence of released neutrophil components, only small amounts of M1 protein were found in bacterial supernatants, whereas larger quantities of M1 protein fragments with different molecular masses were detected when bacteria were incubated with increasing volumes of neutrophil secretion products. The size of the largest M1 protein fragment in comparison to purified M1 suggested that it covers most, if not all, of the extra-cellular part of the M1 protein. With increasing concentrations of neutrophil secretion products M1 protein was further degraded.

To test whether the generated M1 protein fragments were still capable of binding fibrinogen, solubilized streptococcal proteins (10 ng purified M1 protein, AP1 surface proteins released with 100 μl neutrophilic secretion products and 10 ng purified protein H) were run on SDS-PAGE after treatment with the highest volume of neutrophil exudate. They were then transferred onto nitrocellulose and probed with fibrinogen (2 μg/ml). Bound fibrinogen was then immuno-detected with specific antibodies against fibrinogen and a peroxidise-conjugated antibody against rabbit immunoglobulin, as described earlier. E. coli-produced soluble M1 protein binds fibrinogen with high affinity, whereas the closely related protein H shows no interaction with fibrinogen (Åkesson et al., 1994, Biochem. J., 300, 877-886; Berge et al., 1997, J. Biol. Chem., 272, 20774-20781). This was demonstrated in our results, which also showed that the treatment with secreted neutrophil components released two fibrinogen-binding fragments from AP1 bacteria. The molecular masses of these fragments correlated well with the M1 protein fragments seen earlier. Transmission electron microscopy analyses of thin-sectioned AP1 bacteria before and after incubation with neutrophil exudates (100 μl PMN exudate/10⁶ bacteria) revealed that these products efficiently removed the fibrous surface proteins of AP1 bacteria. These hair-like structures represent M protein and the results show that the neutrophil exudates release fibrinogen-binding M1 protein fragments from the bacterial surface.

M1 Protein Triggers the Release of Heparin-Binding Protein (HBP) from PMNs in Human Blood

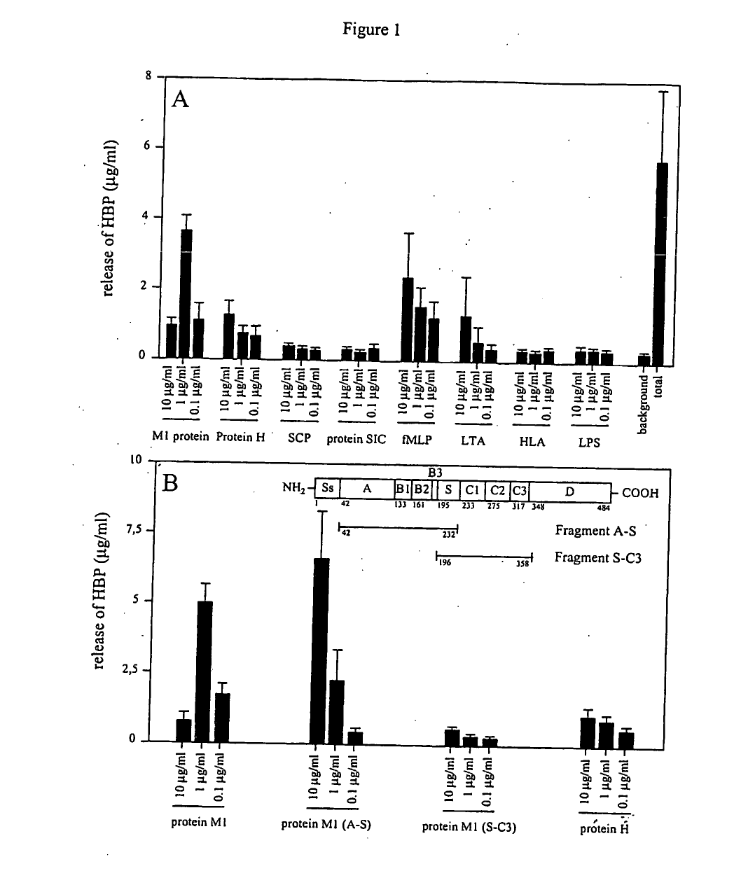

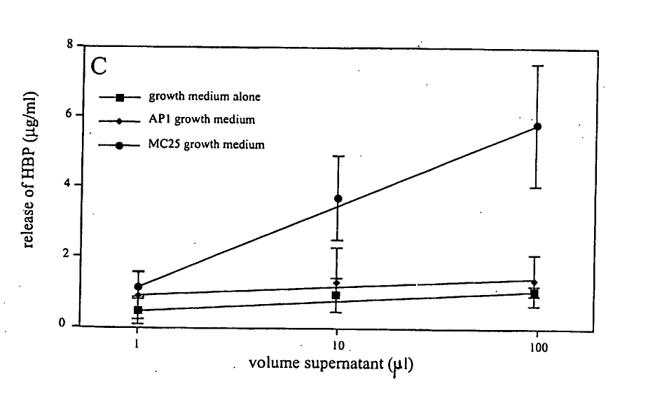

The inflammatory mediator HBP is released by PMNs, the only blood cells that were reported to produce HBP (Edens and Parkos, 2003, Curr. Opin. Haematol. 10, 25-30), and S. pyogenes is known to be a potent inducer of inflammation. The observation that fragments of M1 protein were solubilized by neutrophil proteinases raised the question whether these fragments and/or other S. pyogenes components could enhance the inflammatory response by releasing HBP from PMNs. Soluble streptococcal components were therefore added to human whole blood. FIG. 1A shows that about 63% of the HBP stored in PMNs was mobilized when M1 protein at a final concentration of 1 μg/ml was added to blood. Interestingly, both lower and higher concentrations resulted in less efficient HBP release. Apart from M1 protein, formyl-methionyl-leucyl-phenylalanine (fMLP) and lipoteichoic acid (LTA) evoked secretion of HBP. However, in contrast to the M1 protein-induced release, these effects were dose dependent. Hyaluronic acid (HA), which is part of the streptococcal capsule, and the secreted streptococcal proteins SpeB and protein SIC, did not induce HBP release. Protein H, an IgG-binding surface protein of is AP1 bacteria (Åkesson et al., 1990, Mol. Immunol., 27, 523-531), is structurally closely related to the M1 protein, but does not bind fibrinogen (Åkesson et al., 1994, Biochem. J., 300, 877-886). Only minute amounts of HBP were secreted following the addition of protein H to blood.

To localize the region in the M1 protein that triggers secretion of HBP from PMNs, fragments A-S and S-C3 (Åkesson et al., 1994, Biochem. J., 300, 877-886) derived from the M1 protein (FIG. 1B, top), were tested. FIG. 1B shows that treatment with fragment A-S led to mobilization of HBP, whereas fragment S-C3 had no effect. The results demonstrate that the NH₂-terminal part of the M1 protein is required for HBP release. Previous studies have identified fibrinogen-binding site(s) in the B domains of fragment A-S, albumin-binding sites in the C repeats of S-C3, and IgGFc-binding activity in the S region, which is present in both fragments (Åkesson et al., 1994, Biochem. J., 300, 877-886). The M1 protein and its two fragments are recombinant proteins produced in E. coli.However, also M1 protein produced by S. pyogenes releases HBP, as shown with an isogenic AP1 mutant strain, termed MC25, expressing a truncated M1 protein lacking the COOH-terminal cell wall anchoring motif. This strain has no surface-bound M1 protein, but produces an M1 protein fragment that is secreted into the growth medium (Collin and Olsén, 2000, Mol. Microbiol., 36, 1306-1318). FIG. 1C shows that supernatants of an overnight culture from MC25 bacteria triggered the release of HBP, while culture supematants from AP1 bacteria or growth medium alone did not have this effect. The results demonstrate that soluble M1 protein produced by E. coli or S. pyogenes induces HBP release in human blood.

The Release of HBP from PMNs in Human Blood is Modulated by Signal Transduction Mediators and Extracellular Divalent Metal Ions

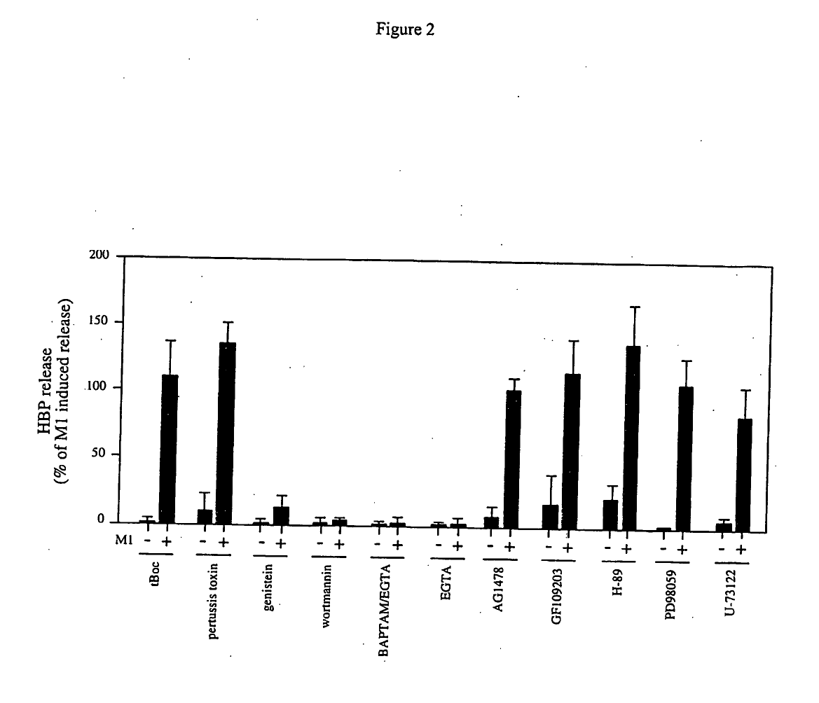

PMNs release their granular content upon cell lysis or by a regulated secretory mechanism involving a sophisticated signal transduction machinery (Borregaard and Cowland, 1997, Blood, 89, 3503-3521). To investigate by which mechanism M1 protein induces mobilization of HBP in human blood, the influence of signal transduction inhibitors on HBP release was analyzed. Theoretically, fMLP contamination of the M1 protein preparation could cause activation of PMNs, and the first substances tested were t-boc-MLP (an fMLP antagonist) and pertussis toxin (an antagonist of Gᵢ protein-coupled seven membrane spanning receptors, to which FMLP receptors belong). As shown in FIG. 2 and Table 1, none of the two components inhibited the release of HBP, implicating that fMLP was not present in the M1 protein preparation and that M1 protein does not act as an fMLP receptor agonist. The next signal transduction inhibitors to be employed were genistein (a tyrosine kinase inhibitor (O'Dell et al., 1991, Nature, 353, 558-560)) and wortmannin (a phosphatidylinositol 3-kinase inhibitor (Cardenas et al., 1998, Trends Biotechnol., 16, 427-433)). These inhibitors abrogate down-stream effects Of β₂ integrin-triggered PMN signaling (Axelsson et al., 2000, Exp. Cell. Res., 256, 257-263), and both blocked the release of HBP almost completely. To study the effect of intracellular and extracellular calcium, cells were incubated with BAPTA (complexing intracellular calcium) and EGTA (complexing extracellular calcium). Like genistein and wortmannin, this treatment inhibited the mobilization of HBP. When EGTA was used in the absence of BATPA, it also blocked HBP release. These results suggest that the binding of M1 protein to PMNs is dependent on divalent metal ions. Other inhibitors which are mainly involved in the signal transduction pathways of G protein-coupled receptors and growth hormone receptors, such as AG1478 (a selective inhibitor of EGF receptor tyrosine kinase (Osherov and Levitzki, 1994, Eur. J. Biochem., 225, 1047-1053)), GF109203 (a protein kinase C inhibitor (Toullec et al., 1991, J. Biol. Chem., 266, 15771-15781)), H-89 (an inhibitor of cAMP-dependent protein kinase (PKA) (Fujihara et al., 1993, J. Biol. Chem., 268, 14898-14905)), PD98059 (an inhibitor of the MAPK pathway (Dudley et al., 1995, Proc. Natl. Acad. Sci. USA, 92, 7686-7689)), and U-73122 (a phospholipase C inhibitor (Smallridge et al., 1992, Endocrinology, 131, 1883-1888)), did not interfere with the secretion of HBP. Taken together, the results show that the release of HBP induced by M1 protein is dependent on the binding of the streptococcal protein to a receptor-like structure located at the neutrophil surface. The data also demonstrate that the binding is dependent on extracellular divalent metal ions.

M1 Protein Precipitates Fibrinogen in Plasma

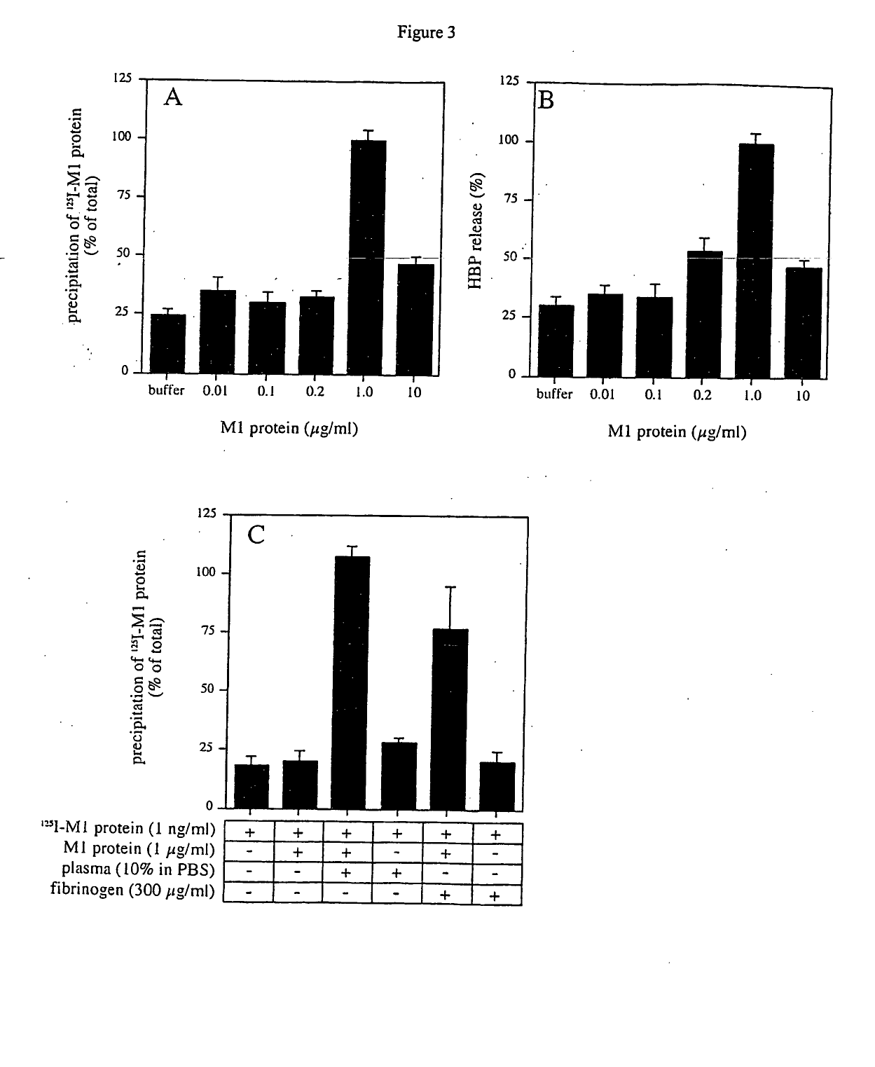

To identify a neutrophil receptor mediating the release of HBP in blood, binding of ¹²⁵I-M1 protein to purified PMNs was tested. However, no significant binding to the PMNs was detected, suggesting that the interaction requires a co-factor, presumably a plasma protein. One of our initial observations was that the addition of M1 protein (at a concentration of 1 μg/ml) to plasma (diluted 1/10) provoked a visible precipitation, while at other concentrations of M1 protein no precipitate was formed in the plasma sample (FIG. 3A). Notably, maximal release of HBP from PMNs was also recorded at a M1 protein concentration of 1 μg/ml blood diluted 1/10 (FIG. 3B), suggesting that M1 precipitation and HBP release are correlated. The finding that M protein forms precipitates in human plasma was reported already in 1965, and was found to be the result of interactions between M protein and fibrinogen (Kantor, 1965, J. Exp. Med., 121, 849-859). The interaction between purified M1 protein and fibrinogen in solution was therefore investigated, and also in this case a precipitate was formed at the same concentrations of M1 protein and fibrinogen as in plasma (FIG. 3C). In contrast, no precipitation occurred when M1 protein was added to fibrinogen-deficient plasma (data not shown). The presence of serine proteinase inhibitors did not influence M1 protein-induced precipitation, indicating that a thrombin-like cleavage of fibrinogen did not cause the precipitation (data not shown). Scanning electron micrographs of the precipitates revealed amorphous aggregation, where individual protein components could not be distinguished. In contrast, plasma clots induced by thrombin showed networks of fibrin fibrils similar to those described previously (Herwald et al., 1998, Nat. Med., 4, 298-302; Persson et al., 2000, J. Exp. Med., 192, 1415-1424). Analysis by transmission electron microscopy of ultra-thin sections at higher resolution showed irregular micro-fibrilar M1 protein/plasma precipitates and highly organized cross-striated thrombin-induced fibrin fibrils. The results show that M1 protein, when added to human plasma in a narrow concentration range, has the potential to trigger plasma precipitation. The precipitate formed is morphologically different from a physiological clot induced by thrombin.

Precipitates of M1 Protein and Fibrinogen Activate PMNs

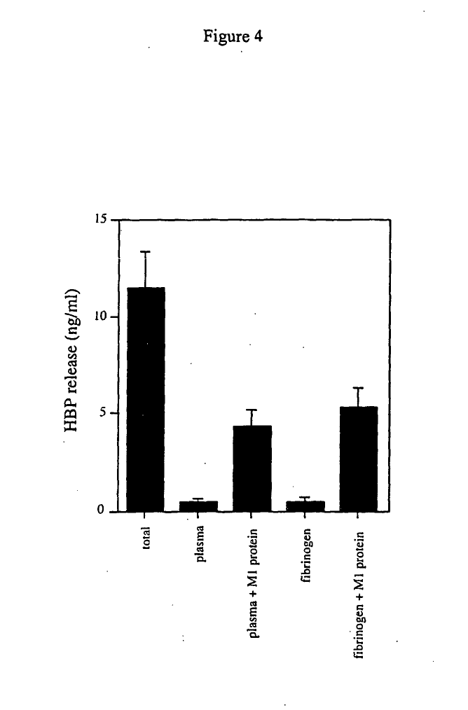

In another set of experiments, we analyzed the interaction between M1 protein/fibrinogen precipitates and PMNs by scanning electron microscopy. The results showed that PMNs reconstituted with a mixture containing M1 protein (1 μg/ml) and human plasma (10% in PBS) formed aggregates that are covered with an amorph proteinous layer, similar to the M1 protein/fibrinogen precipitates seen earlier. No precipitation or aggregation was found when PMNs were reconstituted with plasma in the absence of M1 protein, or when PMNs were treated with M1 protein dissolved in buffer instead of plasma. Purified PMNs incubated with buffer alone were used as a control. Additional experiments with plasma revealed that the aggregation of PMNs in the presence of M1 protein was fibrinogen-dependent (data not shown). The data indicate that the interaction between PMNs and M1 protein/fibrinogen complexes precipitates activates the cells, which results in HBP release. We therefore analyzed whether preformed M1 protein/fibrinogen precipitates are required for PMN activation. M1 protein (final concentration 1 μg/ml) was incubated with fibrinogen (0.3 mg/ml) or with plasma (diluted 1/10) for 30 min. Following centrifugation and washing, the resulting pellets were added to human blood (diluted 1/10) for 30 min and the release of HBP was determined. As a control, fibrinogen and plasma in the absence of M1 protein was treated in the same way. FIG. 4 demonstrates that M1 protein-induced precipitates formed in a fibrinogen solution or in plasma caused HBP release, whereas the controls were negative. Combined the data described in this paragraph show that M1 protein/fibrinogen precipitates bind to PMNs and induce their aggregation and activation, which results in the release of HBP.

M1 Protein-Induced HBP Release is Blocked by a β₂ Integrin Antagonist

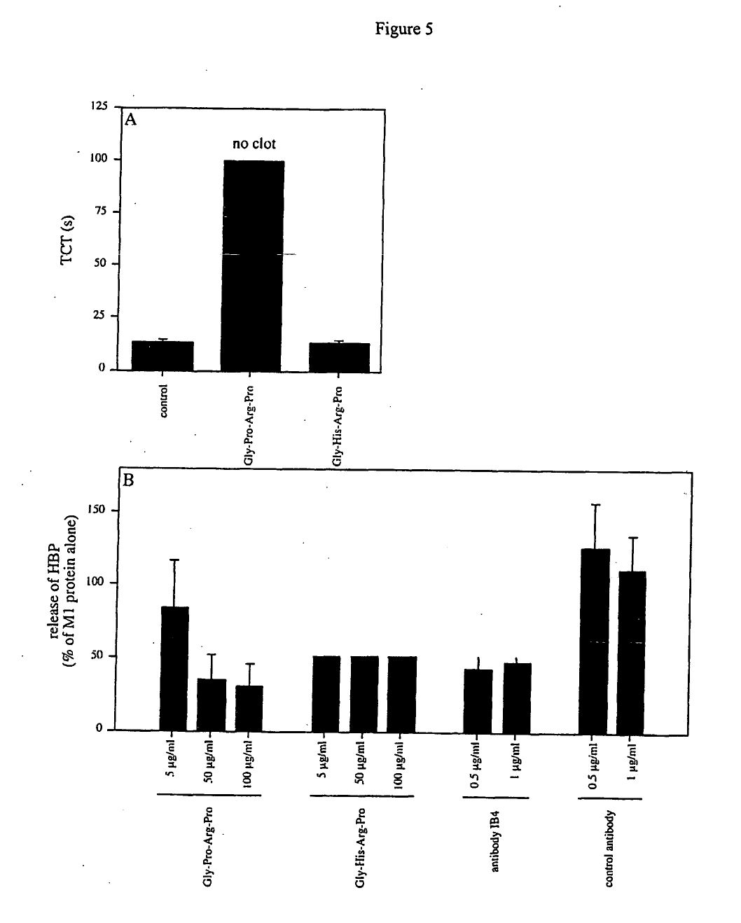

Human fibrinogen binds to PMNs via β₂ integrins (Altieri, 1999, Thromb. Haemost., 82, 781-786) and for CD 11 c/CD 18 the binding site was mapped to the NH₂-terminal region of the Aα chain of fibrinogen. A peptide derived from this region (Gly-Pro-Arg-Pro), has been shown to block adherence of TNF-stimulated PMNs to fibrinogen-coated surfaces, while other peptides from the same region, including Gly-His-Arg-Pro, had no effect (Loike et al., 1991, Proc. Natl. Acad. Sci. USA, 88, 1044-1048). Furthermore, it was demonstrated that antibodies against β₂ integrins inhibit the binding of fibrinogen to activated PMNs, and among these antibodies a monoclonal antibody (IB4) directed against the common β-chain of integrins, was the most potent (Loike et al.,1991, Proc. Natl. Acad. Sci. USA, 88, 1044-1048). Platelet-induced activation of PMNs was also found to be dependent on the interaction between CD11 c/CD18 and the Aα chain of platelet-expressed fibrinogen (Ruf and Patscheke, 1995, Br. J. Haematol., 90, 791-796). As shown for the binding of fibrinogen to PMNs, platelet-induced activation was also inhibited by the Gly-Pro-Arg-Pro peptide and by antibodies to CD11c, whereas the Gly-His-Arg-Pro peptide had no effect. These reports indicate that the binding of PMNs to immobilized fibrinogen (for instance on coverslips or platelets) involves the β₂ integrins leading to an activation of PMNs. Interestingly, Gly-Pro-Arg-Pro not only inhibits the binding of fibrinogen to β₂ integrins, but it also prevents clot formation (Laudano and Doolittle, 1980, Biochemistry, 19, 1013-1019), and FIG. 5A shows that Gly-Pro-Arg-Pro completely blocked thrombin-induced coagulation of normal plasma, while Gly-His-Arg-Pro did not influence the clotting time. It should be emphasized that Gly-Pro-Arg-Pro prevents fibrin-fiber formation by binding to the thrombin exposed polymerization sites of the fibrin molecules (Spraggon et al., 1997, Nature, 389, 455-462). Thus, the effect of Gly-Pro-Arg-Pro on clot-formation is not integrin-dependent. The influence of the two peptides on the interaction between M1 protein and fibrinogen was tested in a competitive ELISA. However, none of the peptides had an effect in these assays (data not shown).

The Gly-Pro-Arg-Pro and Gly-His-Arg-Pro peptides, as well as antibodies to the β₂ integrins (IB4), were also tested for their ability to interfere with the M1 protein-induced secretion of HBP. As shown in FIG. 5B, the addition of Gly-Pro-Arg-Pro to human blood blocked the mobilization of HT3P by M1 protein in a dose dependent manner, and also antibody IB4 directed against the common β-chain of integrins impaired the release. The control substances, Gly-His-Arg-Pro and an unrelated antibody to H-kininogen, did not influence HBP secretion (FIG. 5B). The effect of Gly-Pro-Arg-Pro on M1 protein-induced PMN aggregation was confirmed by scanning electron microscopy analysis. Gly-Pro-Arg-Pro inhibited the aggregation of purified PMNs in a mixture of plasma and M1 protein. In contrast, Gly-His-Arg-Pro had no effect on the aggregation of PMNs. These results support the notion that M1 protein-fibrinogen complexes activate PMNs through β₂ integrin ligation, which triggers the release of HBP. This mechanism appears to be similar to the previously described antibody-mediated cross-linking of CD11b/CD18 that mimics adhesion-dependent receptor engagement causing a massive release of HBP from PMNs (Gautam et al., 2000, J. Exp. Med., 191, 1829-1839).

Intravenous Injection of M1 Protein into Mice causes Severe Lung Lesions that are Prevented by the Administration of a β₂ Integrin Antagonist

So far, HBP has only been identified in humans and pigs (Flodgaard et al, 1991, Eur J. Biochem, 197, 535-547). Before mouse experiments were performed, we investigated whether an HBP homologue is also present in the mouse. To this end, bone marrow cells from mice were isolated and the existence of a murine HBP homologue was demonstrated by RT-PCR analysis and Western blot analysis. RT-PCR amplification of RNA prepared from bone marrow cells was carried out using a primer set derived from the human HBP sequence. Western blot detection was carried out after electrophoresis of human HPB and murine bone marrow lysate immunostained with antibodies against human HBP. A series of animal experiments was then conducted with anaesthetized mice. Three mice received M1 protein i.v. (15 μg/animal); three were treated with a mixture of M1 protein (15 μg/animal) and peptide Gly-Pro-Arg-Pro (400 μg/animal); three with a mixture of M1 protein (15 μg/animal) and peptide Gly-His-Arg-Pro (400 μg/animal); and three with vehicle alone. Thirty minutes after administration the breathing of mice injected with M1 protein or M1 protein plus peptide Gly-His-Arg-Pro was clearly affected as compared to the other mice. The animals were sacrificed and the lungs were removed, stained with hematoxylin and eosin and subjected to light microscopy or analyzed by scanning electron microscopy. A representative lung sample from a mouse injected with buffer only showed intact lung tissue. Lung sections from mice injected with M1 protein, however, demonstrated severe hemorrhage and tissue destruction. These lesions were almost completely prevented when M1 protein was injected together with Gly-Pro-Arg-Pro, even though the tissue remained slightly swollen which is a sign of an ongoing inflammatory reaction. By contrast, application of Gly-His-Arg-Pro could not prevent the M1 protein induced bleeding and tissue destruction. Protein H was injected as a control and analysis of the lung tissue revealed no hemorrhage and the alveoli appeared less swollen. In order to resolve lung lesions at higher magnification, tissue sections were analyzed by scanning electron microscopy. A lung section from a PBS-treated mouse showed no signs of any pulmonary damage. However, injection of the M1 protein resulted in severe leakage of erythrocytes as seen before, but also in the deposition of proteinous aggregates. The morphology of the aggregates resembled the M1 protein-induced amorphous plasma precipitates seen earlier. The lungs of mice injected with M1 protein and Gly-Pro-Arg-Pro contained no precipitates. However, some alveolar swelling and minor leakage of erythrocytes were observed indicating an inflammatory reaction. In contrast, treatment with Gly-His-Pro-Arg did not influence M1 protein-caused lung damage. The injection of protein H did neither cause serious bleeding nor did the tissue appear to be severely inflamed.

In order to quantify the degree of lung affection six randomly chosen lung tissue section from each of the twelve animals were analyzed by electron microscopy, and the ratio of lung area containing protein aggregates versus total lung area was determined. Less than 10% of the lung tissue of animal injected with buffer alone or with M1 protein plus the Gly-Pro-Arg-Pro peptide contained protein aggregates (3±1% and 6±2%, respectively). In contrast, 90% of the lungs of animals treated with M1 protein or a mixture of M1 protein and the Gly-His-Arg-Pro peptide contained protein aggregates (90±2% in both cases). These animal experiments suggest that M1 protein-fibrinogen aggregates activate PMNs via the β₂ integrins, resulting in massive vascular leakage and deposition of protein aggregates in the lung tissue. The results also show that this pathophysiological effect can be blocked when fibrinogen-induced crosslinking of β₂ integrins is prevented by the Gly-Pro-Arg-Pro peptide.

Gly-Pro-Arg-Pro Prevents Vascular Leakage and Lung Damage in Mice Infected with M1 Protein Expressing S. pyogenes Bacteria

In a second series of animal experiments, nine mice were subcutaneously infected with M1 protein expressing S. pyogenes bacteria. Three mice in each group were treated with peptides Gly-Pro-Arg-Pro and Gly-His-Arg-Pro as described in Material and Methods, respectively, while three mice received no treatment. As a control, three mice were given a subcutaneous injection of PBS. Six hours after infection, animals were sacrificed, lungs removed and examined by scanning electron microscopy. Analysis of blood samples from the animals revealed no occurrence of streptococci, indicating that bacteria had not started to disseminate from the site of infection. Electron micrographs of representative lung tissue sections from these animals were obtained. Recovered lungs from mice that received buffer instead of bacteria showed no signs of pulmonary damage. However, mice that were infected with streptococci were suffering from severe lung lesions indicated by massive infiltration of erythrocytes and fibrin deposition. When infected animals were treated with Gly-Pro-Arg-Pro, the lungs appeared to be much less affected, whereas treatment with Gly-His-Arg-Pro failed to prevent pulmonary damage. Lungs from mice infected with streptococci were further analyzed by immuno-staining electron microscopy by using antibodies against M1 protein. This showed that the M1 protein was found in the infiltrated precipitates. In contrast, no M1 protein staining was observed when lungs from non-infected animals were examined. Taken together, these results suggest that in an infectious model, shedded M1 protein is found in the circulation prior to dissemination of bacteria forming precipitates that deposits in the lungs of infected animals.

M1 Protein/Fibrinogen Precipitates are formed in a Patient with Streptococcal Toxic Shock Syndrome and Necrotizing Fasciitis

STSS constitutes a serious complication from a streptococcal infection and is associated with high morbidity and mortality (for a review see (Stevens, 2003, Curr Infect Dis Rep, 5, 379-386). Clinical signs of STSS are acute pain, erythema of the extremity, hypotension, fever, soft-tissue swelling, and respiratory failure (Stevens, 2000, Annu Rev Med, 51, 271-288). As our in vitro and in vivo data imply that some of these symptoms could be caused by the interaction between M1 protein and fibrinogen and the subsequent release of HBP, we analyzed tissue sections from a patient suffering from STSS necrotizing fasciitis caused by infection with an M1 protein-expressing M1T1 strain. A tissue section was sectioned, fixed, stained for M1 protein and fibrinogen and examined by confocal immuno-fluorescence microscopy by using antibodies against human fibrinogen and M1 protein (as described in Materials and Methods). The micrograph revealed large amounts of streptococci found at the epicenter of infection (i.e. fascia) with the M1 protein which was readily detected in these areas. Although some of the M1 protein was found associated with the bacteria, the vast majority of the protein was released from the streptococcal surface. Non-specific staining was ruled out since the M1 protein was not detected in biopsies from distal areas with no or only very low bacterial load. Importantly, the shedded M1 protein was strongly co-localized with fibrinogen at the local site of infection, demonstrating that the amount of released M1 protein that was generated during the course of infection was sufficient to form precipitates with fibrinogen. Taken together the results provide strong evidence that in patients suffering from STSS necrotizing fasciitis, the release of M1 protein from the bacterial surface followed by the formation of M1 protein/fibrinogen precipitates presents an important virulence mechanism.

TABLE 1
Inhibition of M1 protein-induced release of HBP in human blood

| substance | target | effect |
|---|---|---|
| t-boc-MLP | fMLP receptor | no inhibition |
| pertussis toxin | Gᵢ protein-coupled seven | no inhibition |
| | membrane spanning receptors | |
| genistein | tyrosine kinases | full inhibition |
| wortmannin | phosphatidylinositol 3-kinase | full inhibition |
| BAPTA and EGTA | intra- and extracellular calcium | full inhibition |
| EGTA | extracellular calcium | full inhibition |
| AG1478 | EGF receptor tyrosine kinase | no inhibition |
| GF109203 | protein kinase C | no inhibition |
| H-89 | cAMP-dependent protein kinase | no inhibition |
| PD98059 | MAPK pathway | no inhibition |
| U-73122 | phospholipase C | no inhibition |

1-28. (canceled)

29. A method for identifying an anti-streptococcal agent, which method comprises:
(a) providing, as a first component, an isolated streptococcal M protein or a functional variant thereof;
(b) providing, as a second component, isolated fibrinogen or a functional variant thereof;
(c) providing, as a third component, an isolated ∃₂ integrin or a functional variant thereof;
(d) contacting said components with a test substance under conditions that would permit the components to interact in the absence of the test substance; and
(e) determining whether the test substance inhibits the interaction between the components;
thereby to determine whether a test substance is an anti-streptococcal agent; or which method comprises:
(f) providing, as a first component, a streptococcal M protein or a functional variant thereof;
(g) providing, as a second component, fibrinogen or a functional variant thereof;
(h) providing, as a third component, one or more polymorphonuclear neutrophils (PMNs);
(i) contacting said components with a test substance under conditions that would permit the components to interact in the absence of the test substance; and
(j) monitoring any inhibition of the activation of PMNs; thereby to determine whether a test substance is an anti-streptococcal agent.

30. A method according to claim 29 wherein step (i) comprises contacting S. pyogenes, fibrinogen and PMNs in the presence of a test substance.

31. A method according to claim 29 wherein inhibition of the activation of PMNs is monitored by measuring the release of heparin binding protein (HBP).

32. A method according to claim 29 wherein the first component is provided by contacting Streptococcus pyogenes with a protease.

33. A method according to claim 32 wherein the protease is derived from aPMN.

34. A method according to claim 32 wherein the protease is endogenous to S. pyogenes.

35. A method according to claim 29 wherein the streptococcal M protein is the M1 protein of S. pyogenes, a homologue thereof which maintains the ability to form a complex with fibrinogen, or a functional variant of either thereof which maintains the ability to form a complex with fibrinogen.

36. A method according to claim 35, wherein the functional variant is a fragment of the M1 protein of S. pyogenes or a fragment of a homologue thereof.

37. A method according to claim 29, wherein step (e) comprises determining whether the components form aggregates in the presence of the test substance.

38. A test kit comprising:
(a) an isolated streptococcal M protein or a functional variant thereof;
(b) isolated fibrinogen or a functional variant thereof; and
(c) an isolated β₂ integrin or a functional variant thereof;
wherein said test kit is suitable for use in identifying a test substance which is capable of inhibiting the interaction between a streptococcal M protein or a functional variant thereof, fibrinogen and a functional variant thereof and a β₂ integrin or a functional variant thereof; or a test kit comprising:
(d) a streptococcal M protein or a functional variant thereof;
(e) fibrinogen or a functional variant thereof; and
(f) one or more PMNs;
wherein said test kit is suitable for use in identifying a test substance which is capable of inhibiting the interaction between a streptococcal M protein or a functional variant thereof, fibrinogen or a functional variant thereof and PMNs.

39. A test kit according to claim 38 which further comprises one or more buffers.

40. A test kit according to claim 38 further comprising means for determining whether a test substance disrupts the interaction between the components.

41. An anti-streptococcal agent identified by a method according to claim 29.

42. A method of treating an individual suffering from a streptococcal infection, which method comprises:
(a) administering a therapeutically effective amount of an agent identified by a method according to claim 29 to a said individual; or which method comprises:
(b) administering a therapeutically effective amount of an integrin antagonist to a said individual; or which method comprises:
(c) administering a therapeutically effective amount of an inhibitor of the interaction between streptococcal M protein, fibrinogen and β₂ integrin to a said individual; or which method comprises:
(d)(i) identifying an agent that inhibits the interaction between streptococcal M protein, fibrinogen and ∃₂ integrin; and
(ii) administering a therapeutically effective amount of the inhibitor thus identified to a said individual.

43. A method according to claim 42 wherein the integrin antagonist is an anti-integrin antibody, a peptide mimetic or a non-peptide mimetic.

44. A method according to claim 42 wherein the inhibitor of the interaction between streptococcal M protein, fibrinogen and β₂ integrin is a peptide comprising the sequence GPRP.

45. A method according to claim 42 wherein the inhibitor of the interaction between streptococcal M protein, fibrinogen and β₂ integrin is an antibody which specifically binds the B-repeats of S. pyogenes M1 protein.

46. A pharmaceutical composition comprising an inhibitor of the interaction between streptococcal M protein, fibrinogen and ∃₂ integrin identified by a method of claim 29 and a pharmaceutically acceptable carrier or diluent.

47. A method for providing a pharmaceutical composition, which method comprises:
(a) identifying an agent that inhibits the interaction between streptococcal M protein, fibrinogen and β₂ integrin by a method according to claim 29; and
(b) formulating the inhibitor thus identified with a pharmaceutically acceptable carrier or diluent.


SEQUENCE LISTING (11 sequences)

SEQ ID NO: 1 - protein, 484 aa, Streptococcus pyogenes
MAKNNTNRHYSLRKLKTGTASVAVALTVLGAGFANQTEVKANGDGNPREVIEDLAANNPAIQNIRLRYENKDLKARLENAMEVAGRDFKRAEELEKAKQALEDQRKDLETKLKELQQDYDLAKESTSWDRQRLEKELEEKKEALELAIDQASRDYHRATALEKELEEKKKALELAIDQASQDYNRANVLEKELETITREQEINRNLLGNAKLELDQLSSEKEQLTIEKAKLEEEKQISDASRQSLRRDLDASREAKKQVEKDLANLTAELDKVKEDKQISDASRQGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAGKASDSQTPDTKPGNKAVPGKGQAPQAGTKPNQNKAPMKETKRQLPSTGETANPFFTAAALTVMATAGVAAVVKRKEEN

SEQ ID NO: 2 - protein, 4 aa, Artificial Sequence
  Description of Artificial Sequence Synthetic peptide
GPRP

SEQ ID NO: 3 - protein, 4 aa, Artificial Sequence
  Description of Artificial Sequence Synthetic peptide
GHRP

SEQ ID NO: 4 - DNA, 13 bp, Artificial Sequence
  Description of Artificial Sequence Synthetic primer
gggttgttgagaa

SEQ ID NO: 5 - protein, 644 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 6 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 7 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRIMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 8 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSPPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 9 - protein, 1163 aa, Homo sapiens [sequence omitted]

SEQ ID NO: 10 - protein, 769 aa, Homo sapiens
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 11 - protein, 6 aa, Artificial Sequence
  Description of Artificial Sequence Synthetic peptide
KQAGDV